(19) United States
(12) Patent Application Publication
(10) Pub. No.: US 2022/0167069 A1
(43) Pub. Date: May 26, 2022

(54) AUTOMATED ENVIRONMENT MONITORING SYSTEMS AND METHODS

(71) Applicant: Reign RMC, LLC, Richardson, TX (US)

(72) Inventors: Richard J Vanek, Summerland Key, FL (US); Karen D Montrose, Pensacola, FL (US); Rodney C Montrose, Pensacola, FL (US); Martin J Pitzen, Blaine, MN (US)

(73) Assignee: Reign RMC, LLC, Summerland Key, FL (US)

(21) Appl. No.: 17/480,118

(22) Filed: Sep. 20, 2021

Related U.S. Application Data
(60) Provisional application No. 63/080,678, filed on Sep. 18, 2020.

(51) Int. Cl. H04Q 9/00; G01F 23/296; G01F 23/292; G01F 23/14; G01N 33/00

(52) U.S. Cl. CPC H04Q 9/00; G01F 23/296; F16M 13/02; G01F 23/14; G01N 33/0044; G01F 23/292

(57) ABSTRACT

The present invention relates to systems and methods for automated monitoring of an environment enclosed by a manhole cover having a manhole. The automated monitoring of the environment generally includes obtaining a first reading from a first sensor coupled to a monitoring assembly, transferring the first reading from the first sensor to an Information Processor and Cloud Transceiver (IPCT), processing the first reading from the first sensor in the IPCT to create an output defined as an operating condition of the environment and communicating the operating condition of the environment across a communications network and to a server when the reading is flagged. The monitoring assembly is attached to either an in-flow protector dish or a ring with support brackets, to allow easy installation and removal without concern for the weight of a manhole cover.

### CLAIM OF PRIORITY, IDENTIFICATION OF RELATED APPLICATIONS

This Non-Provisional Patent Application claims priority from U.S. Provisional Patent Application No. 63/080,678 filed on Sep. 18, 2020 entitled AUTOMATED MANHOLE COVER WATER DETECTION, to common inventors Richard J. Vanek, et al.

### TECHNICAL FIELD

The present invention generally relates to manhole monitoring. More specifically, the present invention relates to systems and methods for automated monitoring of a manhole.

### PROBLEM STATEMENT

### Interpretation Considerations

This section describes technical field in detail and discusses problems encountered in the technical field. Therefore, statements in the section are not to be construed as prior art.

### Discussion of History of the Problem

Manholes are omni-present worldwide in every city's water and sewer systems, especially in the United States. As an example, New York city has over 350,000 manholes in across its five boroughs. These manhole covers not only provide access to water and sewer systems, they also cover openings to a wide variety of underground tunnels and vaults. In these vaults, tunnels and pipes, water levels fluctuate up and down across of wide range of temperature and weather extremes.

Monitoring water levels is important for early detection of overflow conditions and detecting blockages in these tunnels (and especially in sewer systems). As one may imagine, when a manhole pipe overflows, consequences range from minor rainwater overflow, to serious flooding and biohazards such as raw sewage spillage flooding streets.

Other parameters in these systems require monitoring. For example, the buildup of Hydrogen Sulfide (H2S) gas is created from sewar gas and is extremely corrosive and dangerous to humans. Monitoring H2S levels is important for safe water, sewage, and underground tunnel systems.

Typically, a flow rate of water can be determined by measuring the level of the water where the shape(s) and dimensions of its channel are known using Manning's Equation (also known as Manning formula, Gauckler-Manning formula, or Gauckler-Manning-Strickler formula). Knowing the liquid flow rate is particularly important for monitoring the operation of pipes and manholes in a utility system.

It is well-known that manholes are typically covered with a manhole cover that is typically very heavy (often in excess of several hundred pounds of weight). The weight and dimensions of a manhole cover make it difficult to remove and replace from its collar, where it sites in or above the manhole. Accordingly, when a manhole cover is removed and replaced, if electronics are attached to the manhole cover, they are often damaged.

### TOPICALLY RELATED PUBLICATIONS

Several monitoring systems and methods are known, including:

U.S. Pat. Nos. 7,002,481B1 and 7,768,413 disclose monitoring systems for manhole data, where a monitoring device is placed into a manhole and suspended using brackets permanently attached to a manhole cavity;

U.S. Pat. No. 7,589,630B2 teaches a remote sensing communications system attached to a manhole cover having a special internal antenna therein (its primary function is to detect tampering of the manhole cover);

U.S. Pat. No. 7,598,858B2 discloses a remote monitoring system for a manhole cover having sensors dangling from a main communications and processor element; and

U.S. Pat. No. 8,258,977 discloses a manhole cover for use with a system for transmitting data to an above surface receiver. A monitoring apparatus is attached to a special manhole cover with a center containing an antenna to transmit signals.

Accordingly, there exist a need for systems, devices and methods that facilitate the monitoring of water levels, flow rates, and gasses in a tunnel or pipe covered by a manhole. The present invention overcomes the drawback of equipment damage while providing monitoring the aforementioned pipes and tunnels having manholes.

### SUMMARY

The present invention relates to monitoring of at least one environmental condition—including water level, H2S gas concentration, or flow rates—in a system that incorporates a manhole and manhole cover.

In one aspect, the invention is a monitoring system for closed environments that incorporate a manhole cover (“the monitoring system”). The monitoring system comprises a manhole monitoring assembly having an in-flow protector dish, a first sensor connected to the monitoring assembly, and an IPCT (Information Processor and Cloud Transceiver) coupled to the monitoring assembly.

The first sensor is adapted to monitor at least one physical characteristic of an environment within a system having a manhole cover.

The IPCT obtains a first reading from the first sensor, processes the first reading from the first sensor and compares the first reading with a prestored value and flags the first reading when the first reading is outside the prestored value. The flagged reading represents warnings related to unsafe operation that is transmitted to a user device via a communications network.

A server that is in communication with the IPCT across a communications network receives the first reading and processes the first reading. The server is adapted to generate a first notification for communication to a first user device.

In accordance with various embodiments of the present invention the first sensor is a water distance sensor, or a pressure sensor used to measure the water level below a manhole cover.

The first sensor is a water level sensor using ultrasonic ranging technology, radio ranging technology, light ranging technology, submerged hydrostatic pressure sensor, for example.

The monitoring system further comprises a second sensor, preferably a H2S sensor, and a third sensor, preferably either a temperature sensor or a flow sensor, and a location-determining device.

The monitoring system receives a water level data, a H2S gas level data, a water flow data, and a location data in the IPCT and communicates the water level data, the H2S gas level data, the water flow data and the location data to the server across the communications network.

In an embodiment of the present invention, the monitoring assembly is mounted to a ring with support brackets, with the ring held between a manhole cover and a manhole collar.

In another aspect of the present invention, a method for monitoring a closed environment that incorporates at least one manhole cover. The method obtains a first reading from a first sensor monitoring a water level and transfers the first reading from the first sensor to an IPCT.

The method further processes the first reading from the first sensor in the IPCT to create an output defined as an operating condition of the manhole's environment and communicates the operating condition of the manhole's environment across a communications network and to a server when the first reading is flagged.

A flagged reading is transmitted across the communications network and to a user device. The server generates a notification for a first technician and sends the notification to the user device associated with the first technician.

In one embodiment the method obtains a location data associated with the manhole and may communicate the location data to the server.

Of course, the present is simply a Summary, and not a complete description of the invention.

### BRIEF DESCRIPTION OF THE DRAWINGS

Various aspects of the invention and its embodiment are better understood by referring to the following detailed description. To understand the invention, the detailed description should be read in conjunction with the drawings.

FIG. 1 illustrates an in-flow protector dish to be used for environmental monitoring with the IPCT and a sensor.

FIG. 2 illustrates the in-flow protector dish coupled with a manhole collar and cover where the in-flow protector dish sits below the manhole cover and its rim is held in place between the cover and collar.

FIG. 3 illustrates a monitoring assembly directly coupled to a composite manhole cover (bottom view).

FIG. 4 illustrates a system for monitoring an environment.

FIG. 5 illustrates the system for monitoring an environment having a monitoring assembly coupled to a ring and suspension bracket strips which is held to a collar by the manhole cover.

FIG. 6 is a flow-chart of a time-based monitoring algorithm/method.

### DESCRIPTION OF AN EXEMPLARY PREFERRED EMBODIMENT

### Interpretation Considerations

While reading this section (Description of An Exemplary Preferred Embodiment, which describes the exemplary embodiment of the best mode of the invention, hereinafter referred to as “exemplary embodiment”), one should consider the exemplary embodiment as the best mode for practicing the invention during filing of the patent in accordance with the inventor's belief. As a person with ordinary skills in the art may recognize substantially equivalent structures or substantially equivalent acts to achieve the same results in the same manner, or in a dissimilar manner, the exemplary embodiment should not be interpreted as limiting the invention to one embodiment.

The discussion of a species (or a specific item) invokes the genus (the class of items) to which the species belongs as well as related species in this genus. Similarly, the recitation of a genus invokes the species known in the art. Furthermore, as technology develops, numerous additional alternatives to achieve an aspect of the invention may arise. Such advances are incorporated within their respective genus and should be recognized as being functionally equivalent or structurally equivalent to the aspect shown or described.

A function or an act should be interpreted as incorporating all modes of performing the function or act, unless otherwise explicitly stated. For instance, sheet drying may be performed through dry or wet heat application, or by using microwaves. Therefore, the use of the word “paper drying” invokes “dry heating” or “wet heating” and all other modes of this word and similar words such as “pressure heating”.

Unless explicitly stated otherwise, conjunctive words (such as “or”, “and”, “including”, or “comprising”) should be interpreted in the inclusive and not the exclusive sense.

As will be understood by those of the ordinary skill in the art, various structures and devices are depicted in the block diagram to not obscure the invention. In the following discussion, acts with similar names are performed in similar manners, unless otherwise stated.

The foregoing discussions and definitions are provided for clarification purposes and are not limiting. Words and phrases are to be accorded their ordinary, plain meaning, unless indicated otherwise.

### Description of the Drawings, a Preferred Embodiment

The present invention generally relates to systems and methods for the automated monitoring of an enclosed environment accessed via a manhole having a manhole cover.

With the procurement of present invention, a municipality accesses an innovative environmental monitoring system installable via manholes, to create multiple independent points of monitored data. Together, the data from each monitoring device indicates the functioning characteristics of an environment accessed at each manhole having a monitoring device. When appropriate, a monitoring device triggers an alert the moment a condition is detected that indicates an operation could be compromised and/or an undesirable physical characteristic is detected in the monitored environment.

By a continuous monitoring of environmental parameters (and/or their underlying physical characteristics) such as water level, H2S gas, natural gas, petroleum-derived products such as automobile gas and/or oil, debris, water flow, and temperature, for example, it can be confirmed that the monitored environment is operating properly. As an example of a monitored physical characteristic, a water distance sensor may detect a physical distance to a water surface (and in some cases a water depth). When the physical dimensions of the pipe, tunnel, vault, or other environment are known, a water level can be determined. Then, by setting a sensitivity level, a user can define a safe water level in the environment.

If the water level is detected to exceed the sensitivity level—or is detected to be rapidly rising—then a spillage from the manhole is possible. Accordingly, upon the detection of one of these conditions, a message regarding the detected condition is sent to a cloud service. The cloud service receives and records the monitored data in near real-time (which preferably includes the water level at the point it began increasing, and the time the increase began), and the cloud service then sends a text, e-mail, or voice notification to an owner, manager, or another person responsible, or technician, for example. When it is detected that the water level has decreased to a safe level within the sensitivity level, the data related to this event is sent to the cloud service which may then compute the time period of the water level increase and decrease and can then send a predetermined message conveying that data to an appropriate recipient.

Advantageously, the above features eliminate guesswork as to which of the thousands of manholes associated with a set of water pipes, tunnels, conduits, or vaults may be clogged or blocked when an operator water is reported to be flowing in streets or sidewalks. Further, the data gather can be used to estimate the location and severity of debris blockage, and/or environmentally hazardous leakages. Accordingly, crews can respond immediately to a detected issue rather than physically searching and diagnosing of the source of a problem.

Another benefit of a monitoring system or device on each manhole is the ability to monitor the water flow rate within the manhole using the Manning formula. With the Manning formula, the flow rate is determined by the water level as a function of the shape and size of the tunnel or pipe (or half-pipe) or other structure through which the water is flowing below a manhole. Being alerted of a water flow difference detected between devices coupled to two manholes provides a technician the ability to identify the location of blockages in a pipe (for example), thus saving costly exploratory inspections.

Further, the invention may operate in a gaseous environment, such as a high H2S gas environment, for example, where a device adapted for this environment reports a H2S gas level. H2S gas is corrosive and can lead to early deterioration of metal structure within an environment—including any pipes and even manholes and manhole covers that contain it. Further, H2S gas is hazardous, explosive, and deadly to humans, even at low concentration levels.

Many manholes have an in-flow protector dish (commonly referred to as a dog-dish, herein simply “dish”) mounted on their underside to prevent fluids and debris from rising through the manhole (and perhaps even pushing a manhole cover off from the manhole). A dish is held in place to a manhole collar by the weight of the manhole cover. Preferably, the monitoring system of the invention attaches to the in-flow protector dish and is installed and removed by lifting the in-flow protector dish. This eliminates the potential damage to the monitoring system by removal and insertion of the manhole cover.

An alternative implementation is to have a ring with arms, where the monitoring system is attached to the arms. The ring is held in place between the manhole cover and the manhole collar. This does not prevent the inflow of water to the manhole, but still provides a lightweight, easy to install and remove monitoring system.

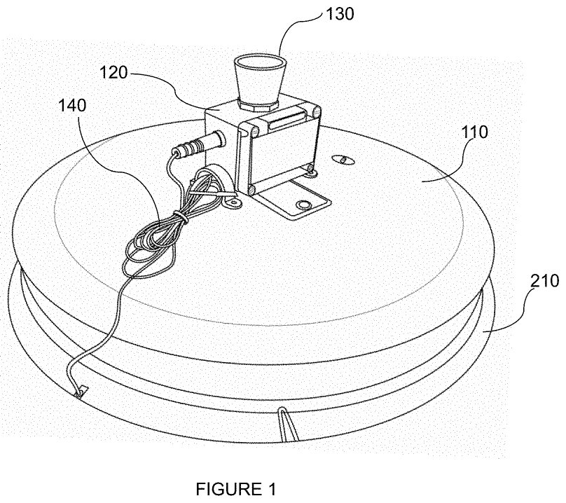

FIG. 1 illustrates an in-flow protector dish 110 (“dish 110”) used for environmental monitoring coupled to a manhole cover 210. The dish 110 is coupled to an Information Processor and Cloud Transceiver (IPCT) 120 and at least one sensor 130 (described below) for monitoring various physical characteristics of an enclosed environment having a manhole, such as the air and liquid level in the environment. Together, an IPCT and the sensor(s) attached to it are referred to as a monitoring assembly. A cable 140 preferably couples the IPCT 120 to an external antenna mounted on an inside lid (not shown, but readily understood by those of skill in the art upon reading the invention) of the dish 110. The antenna may also be external from the manhole unit and above ground.

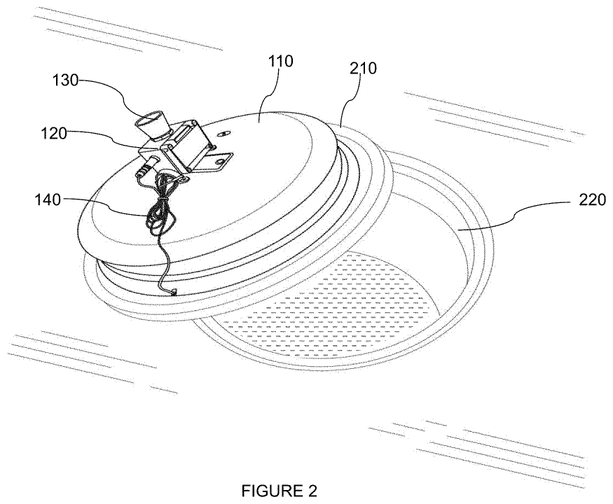

FIG. 2 illustrates the dish 110 coupled to an inner (bottom) surface of a manhole cover 210 that faces downward when positioned on a manhole 220. The manhole cover 210 is in one embodiment an iron manhole cover but may also be made of other materials such as concrete or composite materials, for example.

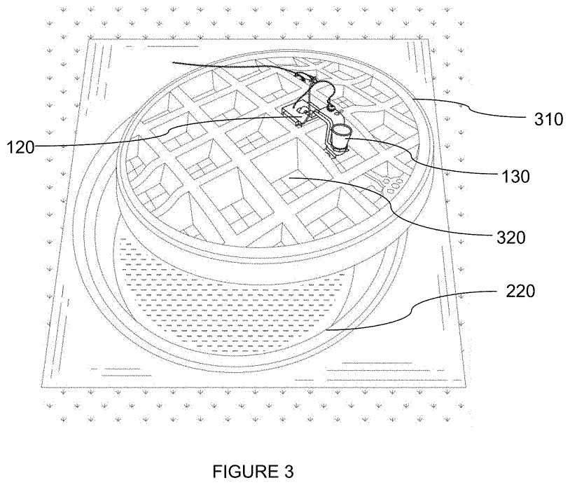

FIG. 3 illustrates an alternative embodiment of the invention, namely a monitoring assembly directly coupled to a manhole cover 310, which is preferably a manhole cover made of a composite material such as a fiber-reinforced polymer composite (also called a “composite manhole cover”). The manhole cover 310 comprises a plurality of waffle-style cells 320 to which an IPCT 120 and a first sensor 130 is attached. The plurality of cells 320 protects the monitoring assembly.

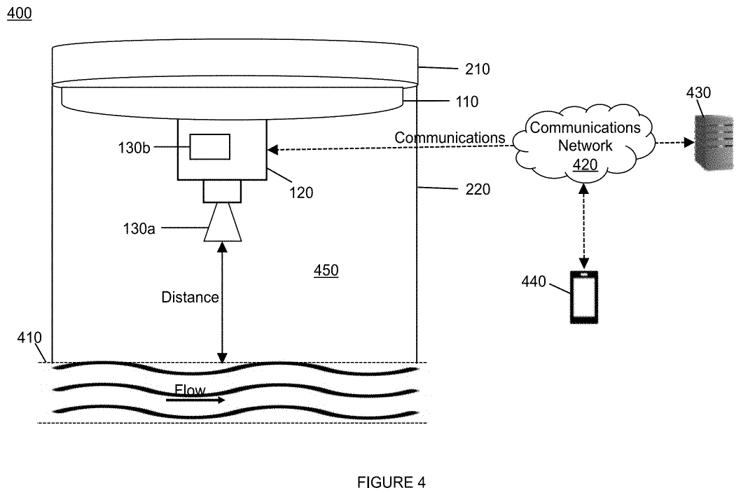

FIG. 4 is a schematic of a system for monitoring an environment having a monitoring assembly (120, 130a, and 130b) coupled to a dish 110. The monitoring assembly may also comprise the manhole cover 210 (shown sitting on top of the dish 110). Preferably, a first sensor 130a and a second sensor 130b monitor different physical characteristic of the environment being monitored, such as water level, H2S gas, natural gas, petroleum-derived products such as automobile gas and/or oil, debris, water flow, or temperature, for example. However, in an alternative embodiment, the first sensor 130a and the second sensor 130b monitor the same physical characteristic and may do so in different ways (such as via a radio signal, sound wave detection, video image monitored with an artificial intelligence trained identification capability, infra-red, or molecule detection sensors, for example).

With specific reference to the manhole cover 210 and the dish 110, the size and type of the manhole cover 210 and dish 110 are selected based on the application of the manhole as well as the type of fluid and/or debris flowing inside a contained environment, such as a pipeline 410 shown in FIG. 4.

Further in FIG. 4, the first sensor 130a is shown mounted to a bottom portion of the IPCT 120, while the second sensor 130b is shown mounted to the side of the IPCT 120. In an alternatively embodiment a sensor is integrated with the IPCT 120. Operationally, the first sensor 130a may detect a water level in the pipeline 410, and the second sensor 130b monitors for any natural gas in the environmental space between the pipe 410 and the manhole cover 210. Data recordings of the readings of the first sensor 130a and the second sensor 130b are periodically or continuously monitored by the IPCT 120 and in the event a reading exceeds pre-set level (referred to a “flag”, “flagging” or as being “flagged”), the readings are then transmitted to a server 430 (referred to as event-based monitoring). Alternatively, or in addition to event-based monitoring, data regard detected environmental conditions and/or physical characteristics are communicated to the server 430 at predetermined times or predetermined time-intervals (referred to as temporal monitoring and periodic monitoring, respectively).

In selected embodiments the first sensor 130a is an ultrasonic sensor, a hydrostatic pressure sensor, a radar distance sensor, or a Light Detection and Ranging (LIDAR) sensor, for example. Alternatively, the first sensor 130a may be selected to comprise a water level sensor using ultrasonic ranging technology, a water level sensor using radio ranging technology, a water level sensor using light ranging technology, or a water level sensor using a submerged hydrostatic pressure sensor, for example. Preferably, the first sensor 130a is a Maxbotix® MB7328-131.

In selected embodiments, a second sensor 130b detects levels of H2S in an environment 450 underneath the manhole 220. Preferably, the second sensor 130b is an electrochemical gas sensor such as SpecSensora® 3SP_H2S_50.

The system 400 further comprises additional sensors including a third sensor, fourth sensor, fifth sensor, sixth sensor, etc. (not shown). Preferably, a third sensor is a temperature sensor or a flow sensor.

The IPCT 120 comprises a transceiver, and a processor such as a PLC (programmable logical controller) or microprocessor. It is the processor of the IPCT 120 that receives and analyzes each reading provided by each sensor, whereas the transceiver communicates with the server 430 such as a remote server which could be a cloud server coupled wirelessly across a network as shown and described in FIG. 4. One preferred transceiver is a SIMCOM® SIM7000A, and a preferred IPCT is the ReignGauge™ from Reign RMC®.

The server 430 communicates with a user device 440 through a communications network 420, which is preferably a wireless communications network such as a Long Term Evolution (LTE), Fifth Generation Networks (5G), Wi-Fi networks and Long Range (LoRa) networks, for example. Of course, the communications network 420 comprises a wireline and/or fiber-optic based communications and data transmission. The user device 440 is a portable electronic device that can communicate with the server 430 such as a laptop, smart phone, tablet, portable computer, pager or any other computing device including similar hardened and field-specific devices.

If a reading is flagged by the IPCT 120, the flagged reading is then transmitted across the communications network 420 to the server 430. The server 430 or the IPCT 120 may accordingly send readings, statuses, warnings, and other data to the user device 440.

In one embodiment, one or more readings from the plurality of sensors 130 are associated with safe functioning, threshold range(s), and unsafe conditions that are stored or loaded to the IPCT 120. If the one or more readings from the first sensor 130 is outside of a desired/prestored value, then the one or more readings are flagged by the processor in the IPCT 120. After flagging, the IPCT 120 sends one or more sensor readings, statuses, warnings or other data to the server 430 and/or the user device 440.

The IPCT 120 may comprise a memory for storing a temporal or time tagged recording of the one or more sensor readings. These readings may be compared with a threshold range(s) to detect unsafe operating conditions in the environment 450 via a processor integrated with the IPCT 120. After a comparison, the one or more sensor readings, along with their respective time tags may be sent to the server 430 and/or the user device 440. Time tagged readings from a plurality of sensors reduces false warnings generated by the monitoring system.

In yet another alternative embodiment, a comparison of values is done at both the IPCT 120 and the server 430. At least one sensor level threshold is specified on the server 430 which sends it to the IPCT 120, whereby the IPCT 120 does a comparison internally. Readings, including periodic readings, can be compared to one or more threshold levels on the server 430. Some environment parameters, such as flow rate—for power consumption, processing power, or communication channel reasons—may be preferably calculated on the server 430. Then, when a threshold is violated, an appropriate warning is communicated.

Additionally, the server 430 stores parameters for the pipeline 410—such as shape, width, depth, surface material (to determine the Strickler value), slope, and use the well-known Manning formula to determine the flow rate of water in an open channel of the pipeline 410 based on the measured water height. The calculated flow rates can be compared to preset values and alerts and alarms sent to the user device 440 and displayed on the server 430 when these are exceeded.

Alternatively, the IPCT 120 sends the one or more sensor readings, statues, warnings, or other data to the server 430 and/or the user device 440 at a predetermined interval of time. The IPCT 120 may send readings from all of the plurality of sensors present in the monitoring assembly at a predetermined interval, to notify all operating conditions of the manhole to the server and/or the user device. Further, the IPCT 120 may combine the readings from the plurality of sensors with the time at which the readings were recorded by the plurality of sensors, and then send the combined reading to the server and/or the user device at a predetermined interval of time.

The IPCT 120 may use a location-determining/location-aware device. For example, a global positioning system (GPS) may be integrated into the IPCT 120 or provided separately. Alternatively, the monitoring assembly may be geo-tagged.

Of course, although reference is made to monitoring a manhole having a single IPCT associated therewith, it is understood and appreciated by those of ordinary skill in the art upon reading this disclosure that multiple manholes may attach to or otherwise communicate with a single IPCT. Further, many manholes and IPCTs operating independently and/or in conjunction with each other may be communicatively coupled to one or more networks.

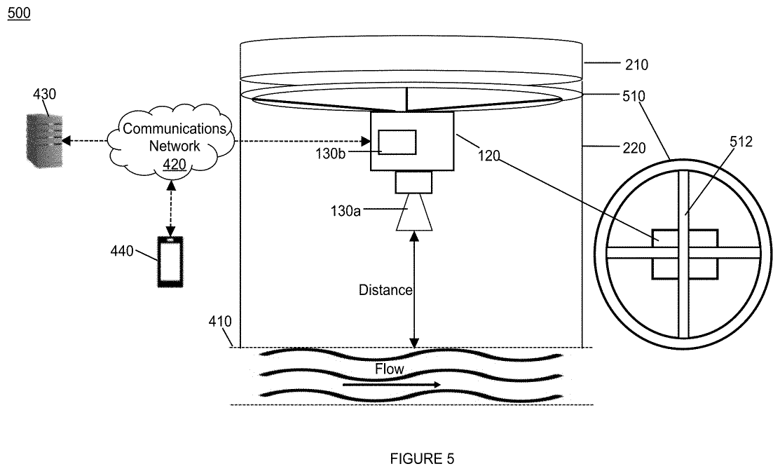

FIG. 5 illustrates a system 500 for manhole monitoring having the monitoring assembly coupled to a ring and suspension bracket strips 510 held to a manhole collar (not shown). The embodiment shown in FIG. 5 is used for manholes when there is no concern about ground water entering the manhole.

Here, the dish 110 is replaced with an assembly 510 comprising a ring 510 (held between the manhole cover 210 and the collar), and brackets 512 that crisscross the ring 510. The IPCT 120 is mounted to the brackets, suspended above the manhole 220. Preferably, the ring 510 and brackets 512 are constructed out of a non-corrosive material such as a plastic material or stainless steel, for example, to make them resistant to the effects of H2S gas.

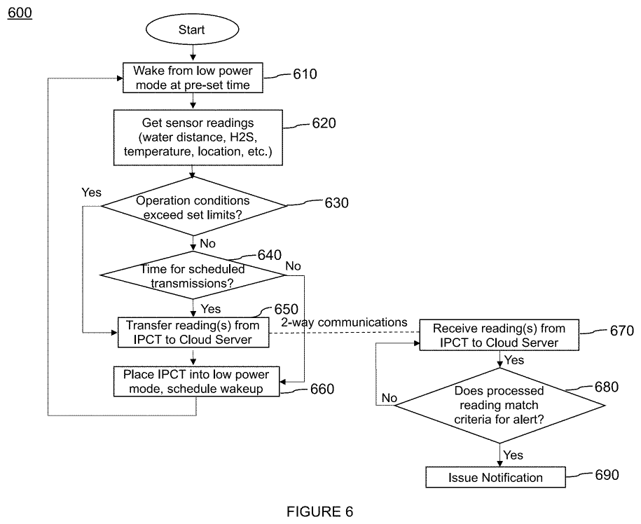

FIG. 6 is a flow-chart of a Time-based Monitoring Algorithm 600. The Time-based Monitoring Algorithm 600 begins in a Wake Act 610, in which an IPCT coupled to a plurality of receivers wakes from a low power mode at a preset time or alternatively after a predetermined time interval. The Wake Act 610 may comprise a low power ‘steady state’ where the Time-based Monitoring Algorithm 600 awaits the receipt of a reading as a discrete packet(s) of data.

Next, in a Get Sensor Readings Act 620, each of the sensors coupled to the IPCT are read to determine the physical characteristics of: water level, H2S gas levels, water flow rate, temperature, and pressure.

Following the Get Sensor Readings Act 620, an Operating Condition Query 630 begins. The Operating Conditions Query 630 asks if the reading obtained indicates that the manhole is operating safely based on the data received. If the physical characteristic(s) of the reading exceed a safe operating range, as indicated by the “Yes” path, then the Time-Based Monitoring Algorithm 600 jumps to a Transfer Readings Act 650 (discussed below).

If, however, the reading analyzed in the Operating Condition Query 630 is within of a safe range, then the Time-Based Monitoring Algorithm 600 proceeds along the “No” (“not exceeded”) path to a Scheduled Transmissions Query 640.

The Transmissions Query 640 asks if it is the appropriate time for a transmission. If it is NOT the appropriate time for a transmission, as indicated by the “No” path, then the Time-Based Monitoring Algorithm 600 jumps to a Low Power Act 660 (described below).

If, however, it is the appropriate time for a transmission in the Transmissions Query 640, then the Time-Based Monitoring Algorithm 600 proceeds along the “Yes” path to a Transfer Readings Act 650. In the Low Power Act 660, the IPCT 120 is placed in a low power rest state to conserve energy. Then the Time-Based Monitoring Algorithm 600 returns to the Wake Act 610.

In a Receive Reading(s) Act 670, a server, such as server 430, receives readings from the IPCT comprising at least one reading from at least one sensor. Next, a Matching Query 680 begins. The Matching Query 680 asks if the reading(s) received a flag or criteria that indicate that the manhole is NOT operating safely or that there's an ‘event of note’ (examples: high water level; rapid increase in water level; high rates of flow). If the physical characteristic(s) of the reading are within an ALERT operating range, as indicated by the “Yes” path, then the Time-Based Monitoring Algorithm 600 issues an alert notification in an Issue Notification Act 690.

If, however, the reading analyzed in the Matching Query 680 is within a safe range, and the Time-Based Monitoring Algorithm 600 proceeds along the “No” path to return to the Receive Reading(s) Act 670.

Although the invention has been described and illustrated with specific illustrative embodiments, it is not intended that the invention be limited to those illustrative embodiments. Those skilled in the art will recognize that variations and modifications can be made without departing from the spirit of the invention. Therefore, it is intended to include within the invention, all such variations and departures that fall within the scope of the appended claims and equivalents thereof.

What is claimed is:

1. A monitoring system for an enclosed environment accessed by a manhole, comprising:
a monitoring assembly comprising
an in-flow protector dish,
a first sensor adapted to monitor at least one physical characteristic of an environment; and
an Information Processor and Cloud Transceiver (IPCT) coupled to the monitoring assembly;
the IPCT
obtains a first reading from the first sensor,
processes the first reading from the first sensor and compares the first reading with a prestored value, and
flags the first reading when the first reading is outside the prestored value;
a server in communication with the IPCT across a communications network, the server for receiving the first reading and for processing the first reading; and
the server is adapted to generate a first notification, the first notification for communication to a first user device.

2. The monitoring system of claim 1 wherein the flagged reading represents warnings related to unsafe operation that is transmitted to a user device via the communications network.

3. The monitoring system of claim 1 wherein the first sensor is a water level sensor using ultrasonic ranging technology.

4. The monitoring system of claim 1 wherein the first sensor is a water level sensor using radio ranging technology.

5. The monitoring system of claim 1 wherein the first sensor is a water level sensor using light ranging technology.

6. The monitoring system of claim 1 wherein the first sensor is a water level sensor using a submerged hydrostatic pressure sensor.

7. The monitoring system of claim 1 further comprising a second sensor, the second sensor being a H2S gas detection sensor.

8. The monitoring system of claim 1 further comprising a third sensor, the third sensor being either a temperature sensor or a flow sensor.

9. The monitoring system of claim 1 further comprising a location-aware device.

10. The monitoring system of claim 1 wherein the IPCT further:
receives a water level data,
receives a H2S gas level data,
receives a water flow data,
receives a location data; and
communicates the water level data, the H2S gas level data, the water flow data and the location data to the server across the communications network.

11. The monitoring system of claim 1 wherein the monitoring assembly is mounted to a ring with support brackets, with the ring held between a manhole cover and a manhole collar.

12. A method for monitoring an environment accessed via a manhole, the method comprising:
obtaining a first reading from a first sensor, the first sensor being coupled to an in-flow protector dish;
transferring the first reading from the first sensor to an Information Processor and Cloud Transceiver (IPCT);
processing the first reading from the first sensor in the IPCT to create an output defined as an operating condition of the environment; and
communicating the operating condition of the environment across a communications network and to a server when the first reading is flagged.

13. The method of claim 12 wherein the flagged reading is transmitted across the communications network to a user device.

14. The method of claim 12 further comprising obtaining a location data associated with the manhole and communicating the location data to the server when the first reading is sent to the server.

15. The method of claim 12 wherein the server generates a notification for a first technician and sends the notification to the user device associated with the first technician.
